(19) United States
(12) Patent Application Publication
(10) Pub. No.: US 2009/0081683 A1
(43) Pub. Date: Mar. 26, 2009

(54) Kits and Methods for Assessing the Coenzyme Q Reducing Status of a Patient, Including a Patient Ingesting a Statin

(75) Inventors: Robert P. Ricciardi, Kennett Square, PA (US); Bernard L. Kasten, JR., Naples, FL (US); Harold H. Harrison, Lewisburg, PA (US)

Correspondence Address: BRANDY C HILL, P.O. BOX 951121, LAKE MARY, FL, 32795, US

(73) Assignee: GeneLink, Inc., Longwood, FL (US)

(21) Appl. No.: 12/236,607

(22) Filed: Sep. 24, 2008

Related U.S. Application Data
(63) Continuation-in-part of application No. 12/095,399, filed on May 29, 2008.
(60) Provisional application No. 60/740,620, filed on Nov. 29, 2005.

(51) Int. Cl. C12Q 1/68

(52) U.S. Cl. 435/6

(57) ABSTRACT

The disclosure relates to kits and methods for assessing whether an individual is likely to benefit from nutritional supplementation with coenzyme Q and, more particularly, a reduced form of coenzyme Q. The methods involve assessing occurrence of a polymorphism in the gene encoding NQO1 in the individual. Individuals homozygous for the polymorphism will receive optimal benefits from supplementation with the reduced form of coenzyme Q. The disclosure further relates to methods for predicting and assigning a coenzyme Q redox status phenotype based on assessment of an individual's genome for a polymorphism in the gene encoding NQO1.

### CROSS-REFERENCE TO RELATED APPLICATIONS

This application is a Continuation-in-Part of U.S. patent application Ser. No. 12/095,399 filed May 29, 2008, which is the national stage application of PCT/US06/45628 filed Nov. 29, 2006, now pending and expressly incorporated herein in its entirety, which is further a non-provisional of U.S. Provisional Application Ser. No. 60/740,620, and is entitled to priority pursuant to 35 USC §120; 35 USC § 365(c), and 37 CFR §1.78. Priority is further claimed under 35 USC §119(e) to U.S. Provisional Application Ser. No. 60/740,620 filed Nov. 29, 2005, also expressly incorporated herein in its entirety.

### STATEMENT REGARDING FEDERALLY-SPONSORED RESEARCH OR DEVELOPMENT

Not applicable.

### FIELD OF THE INVENTION

The disclosure relates generally to the field of physiological and nutritional assessment of individuals. The disclosure further relates to methods for predicting and assigning a coenzyme Q redox status phenotype based on assessment of an individual's genome for a polymorphism in the gene encoding NQO1.

### OBJECTS OF THE INVENTION

It is an object of the invention to provide a method for determining or predicting the redox ratio of reduced CoQ to oxidized CoQ (QH2:Q redox status) of an individual based on assessment of the individual's genome for a polymorphic form of the gene encoding NQO1.

It is an additional object of the invention to determine the QH2:Q redox status phenotype of an individual by assessment of the individual's genome for a polymorphic form of the gene encoding NQO1.

It is also an object of this invention to advise or provide the most ideal form of CoQ supplementation, whether in reduced form (ubiquinol, also designated QH2) or the more commonly available—and more economical-commercial CoQ product in oxidized form (ubiquinone, also designated Q) by assessment of the individual's genome for a polymorphic form of the gene encoding NQO1.

It is a further object of this invention to advise or provide the most ideal form of CoQ supplementation to individuals undergoing treatment with a statin.

The aforementioned objects of the invention are not intended as limiting on the scope of the invention disclosed herein. Likewise, other and further objects of the invention may be apparent or readily concluded from the detailed description of the invention which follows.

### BACKGROUND OF THE INVENTION

The enzyme DT-diaphorase is also known as NAD(P)H:quinone oxidoreductase 1 or NQO1 (after its genetic designation Nqol). Among the known roles of enzyme NQO1 is conversion of the oxidized form (ubiquinone) of coenzyme Q (CoQ) to its reduced form (ubiquinol). This is an obligate two-electron reduction. CoQ is known to exhibit physiologically significant antioxidant activity in humans and other animals. More particularly, it is the reduced form of CoQ that exhibits antioxidant activity, and exhibition of such activity often converts the CoQ to its oxidized state. Activity of the NQO1 enzyme (or another CoQ-reducing enzyme) serves to recycle oxidized CoQ to the useful reduced CoQ form in the body. (Beyer, Molec. Aspects Med. 1994; 15:s117-s129).

CoQ has been identified as a physiologically significant nutrient. Nutritional supplements containing CoQ in a variety of forms and amounts are commercially available. Various compositions containing CoQ have been disclosed. (e.g., U.S. Pat. No. 6,184,255; 6,740,338).

Prior to the present invention, it has been difficult to determine which individuals would benefit most from nutritional supplementation with CoQ or to determine the form of CoQ that such individuals should employ. The present invention provides kits and methods for identifying individuals who will benefit from CoQ supplementation. The present invention further provides a predictor or indicator of the QH2:Q redox status of an individual (a difficult measure to obtain) by assessment of the individual's genome for the NQO1*2 polymorphism (a simple assessment).

Most, if not all, human genes occur in a variety of forms which differ in at least minor ways. Heterogeneity in human genes is believed to have arisen, in part, from minor non-fatal mutations that have occurred in the human genome over time. In some instances, differences between alternative forms of a gene are manifested as differences in the amino acid sequence of a protein encoded by the gene. Some amino acid sequence differences can alter the reactivity or substrate specificity of the protein. Differences between the alternative forms of a gene can also affect the degree to which (if at all) the gene is expressed. Known heterogeneities include, for example, single nucleotide polymorphisms (i.e., alternative forms of a gene having a difference at a single nucleotide residue). Other known polymorphic forms include those in which sequences of larger (e.g., 2-1000 residues) portions of a gene exhibits numerous sequence differences and those which differ by the presence or absence of a portion of a gene. However, many heterogeneities that occur in humans appear not to be correlated with any particular phenotype. Such was the case with respect to the NQO1*2 polymorphism prior to the inventors' discovery. Specifically, the inventors have correlated the NQO1*2 polymorphism with an individual's QH2:Q redox status phenotype. As disclosed by the inventors, individuals who are homozygous, or advantaged, for the wild-type form of the gene encoding NQO1 (i.e., NQO1*1/*1 or +/+) are expected to exhibit optimal QH2:Q redox status. Heterozygous individuals (i.e., NQO1*1/*2 or +/−) are expected to have a lower ratio of QH2:Q than advantaged individuals. And, individuals who are homozygous for the polymorphic form of the gene encoding NQO1 (i.e., NQO1*2/*2) are expected to have an even lower ratio of reduced CoQ to oxidized CoQ than even heterozygous individuals and a diminished or significantly lower ratio compared to phenotypically advantaged individuals. For individuals undergoing a statin treatment, these methods are particularly valuable because of the effect statins have on an individual's CoQ levels.

Making an individual aware of his or her expected QH2:Q redox status has an important advantage inasmuch as an individual who has or is expected to have a lowered (as indicated by the NQO1*1/*2 genotype) or significantly lowered (as indicated by the NQO1*2/*2 genotype) QH2:Q redox status will benefit from CoQ supplementation. Furthermore, supplementation with the most available form of CoQ (the reduced form, ubiquinol) is most suited to these individuals. Nutritional genomics (coined “nutrigenomics”), is the science that studies how a person's diet or supplemented diet interacts with or as a function of his or her genotype to influence health and wellness. Nutrigenomics is an emerging and promising science. The goal of nutrigenomics studies is to understand the relationship between a person's nutrition and his or her genetic predisposition to certain conditions. The present invention addresses the need for a customized nutritional supplementation regimen tailored for an individual's genetic makeup to promote and preserve health. Nutrigenomics changes nutrition and nutritional supplementation from the subjective to the objective.

### BRIEF SUMMARY OF THE INVENTION

The invention relates to a method of assessing the coenzyme Q reducing status of an individual, including an individual undergoing a statin treatment. More particularly, by the inventors' methods, an individual's QH2:Q redox status can be identified or reliably predicted by assessment of the individual's genome for a polymorphic form of the gene encoding NQO1.

1. Advantages of CoQ

As previously mentioned, CoQ is a physiologically significant nutrient. The advantages of CoQ are many.

CoQ was first identified in 1957 and became a subject of interest as a potential treatment for cancer beginning as early as 1961.

As an antioxidant, CoQ relieves oxidative stress and inflammation.

One way in which oxidative stress is received is on the body's largest organ, the skin, by exposure to ultra-violet (UV) radiation. Skin is the largest and most visible organ of the human body, and is also among the tissues most exposed to environmental stresses, hazards, and pathogens. Skin is a multi-layered tissue, primarily composed of the epidermis and dermis, and includes several accessory structures, such as sweat glands, sebaceous glands, and hair follicles. Skin serves many functions. For example, it is a protective barrier to external insults (e.g., heat, chemicals, bacteria), is involved in thermoregulation, inhibits dehydration, and performs sensory functions. Skin is also a bioreactor that produces various hormones and lipids that enter the body's circulation. A variety of immune cells function in skin as a first line of defense against bacterial or viral invasion and to maintain immune surveillance in skin and nearby body tissues. For these reasons, establishment and maintenance of good skin health is important to human health. CoQ has been shown to relieve oxidative stress in skin tissue. CoQ additionally has aesthetic benefits by reason of the fact that CoQ has been shown to reduce signs of photo-aging of the skin caused by UV exposure. CoQ additionally protects against damage caused by oxidative stress in other tissues.

CoQ is believed to have both chemoprotective and chemopreventive attributes. For that reason, a diet rich in fruits and vegetables is considered healthful because fruits and vegetables contain compounds that induce detoxification enzymes (including NQO1). (Fahey, J. W., et al., Broccoli Sprouts: An Exceptionally Rich Source of Inducers of Enzymes that Protect Against Chemical Carcinogens, Proc. Natl. Acad. Sci. 1997; 94:10367-10372).

CoQ supplementation has shown promising outcomes in connection with lowering hypertension without significant side effects.

Significantly, CoQ is an essential cofactor in the mitochondrial electron transport pathway, and is necessary for ATP production. In that role, CoQ acts as a mobile electron carrier, transferring electrons from complex I (NADH coenzyme Q reductase) to complex III (cytochrome bc₁ complex) or from complex II (succinate dehydrogenase) to complex III.

CoQ deficiency has been implicated in a multitude of pathologies including heart failure, hypertension, Parkinson's disease and malignancy. In light of this, supplementation with CoQ in accordance with the inventors' methods is an advisable precaution in maintaining health and wellness.

CoQ is well tolerated by the vast majority individuals. However, a small number of individuals may exhibit symptoms of allergic reaction to CoQ supplementation. Indications of an allergic reaction include shortness of breath and skin irritation. There are no other known precautions for CoQ supplementation. Known side effects of CoQ supplementation are mild and include mild insomnia, skin rash, and nausea.

Recommended supplementation dosages are known in the art. Supplementation dosages are indicated between 5 and 2,400 milligrams per day. Supplementation with CoQ has been shown well-tolerated between levels of 300 and 2,400 milligrams per day.

2. Effect of Statins on CoQ Levels

Concentrations of CoQ in plasma are reduced by statins. Studies performed by others in support of this point suggest that CoQ concentrations are reduced by as much as half in individuals undergoing statin treatment within a period of 14 days following the start of treatment. (Rundek, T., et al., Atorvastatin Decreases the Coenzyme Q10 Level in the Blood of Patients at Risk for Cardiovascular Disease and Stroke, Arch. Neurol. 2004; 61:889-92). For this reason, the inventors' methods are particularly relevant. At present, there is no ready assessment for CoQ concentrations or QH2:Q redox status. However, by practicing the invention disclosed herein, an individual's QH2:Q redox status can be readily and economically determined or predicted.

3. QH2:Q Measuring Challenges

Because CoQ is continually reduced and oxidized, accurate redox ratio measurements are difficult to obtain. The methods used in connection with the experiments performed in support of this disclosure are believed to be the most sensitive, sophisticated and accurate methods presently known. The methods used in support of this disclosure are described by Tang, P. H., et al., Measurement of Reduced and Oxidized Coenzyme Q9 and Coenzyme Q10 Levels in Mouse Tissues by HPLC with Coulometric Detection, Clin. Chim. Acta. 2004; 341(1-2):173-84 and Miles, M., et al., Age-related Changes in Plasma Coenzyme Q10 Concentrations and Redox State in Apparently Healthy Children and Adults, Clin. Chim. Acta. 2004; 341(1-2): 139-44.

While these methodologies are available, their large-scale use is impractical. It is further impractical as a routine clinical test. For that reason, the inventors' methods of predicting QH2:Q redox status based on genetic assessment for a single readily-detectable polymorphism are useful and practical.

The objects (and appurtenant benefits) of the present invention are set forth above. By practicing the methods disclosed herein, one skilled in the art can readily and reliably predict the QH2:Q redox status of an individual and advise appropriate CoQ supplementation by:
(a) obtaining a biological sample from the individual;
(b) assessing the individual's genome for absence or presence of one or two copies of the NQO1*2 polymorphism;
(c) predicting the QH2:Q redox status of the individual based on absence or presence of one or two copies of the NQO1*2 polymorphism;
(d) advising an individual whose genome comprises one or two copies of the NQO1*2 polymorphism to employ a composition comprising CoQ and, more preferably, the reduced form of CoQ (ubiquinol);
(e) providing the individual with an instructional material supplying information relative to the genomic assessment and advised nutritional support; and
(f) applying the methods to individuals undergoing treatment with a statin.

### BRIEF DESCRIPTION OF THE SEVERAL VIEWS OF THE DRAWINGS

The foregoing summary, as well as the following detailed description of the preferred embodiments of the invention, will be better understood when read in conjunction with the appended drawings. The invention is not, however, limited to the arrangements and instrumentalities shown.

Table 1 is titled Study Participant Genotypes and is prepared using data collected in connection with the experiments performed at the inventors' direction in support of this disclosure. This table indicates the Sample ID No. assigned to the blood samples collected from the respective study participants. Table 1 further indicates the participants' NQO1 genotypes. As discussed below, the kit described in U.S. Pat. No. 6,291,171 (2001, Ricciardi, et al.) was employed to collect biological samples from the study participants for purposes of determining genotypes.

Table 2 is titled Participant Demographics and is prepared using data collected in connection with the experiments performed at the inventors' direction in support of this disclosure. This table indicates basic demographic information relating to the study participants; namely, age and gender. It is noted that each of the study participants is female. This does not represent a study parameter. Rather, each member of the pool of potential volunteer study participants was female. Importantly, however, QH2:Q redox status is not affected by gender. (Miles, M. V., et al., Plasma Coenzyme Q10 Reference Intervals, But Not Redox Status, are Affected by Gender and Race in Self-Reported Healthy Adults, Clin. Chem. Acta. 2003; 332: 123-132).

Table 3 is titled Assessment Data and is prepared using data collected in connection with experiments performed in support of this disclosure. The data compiled in Table 3 indicates the optimal QH2:Q redox status exhibited by individuals whose genome does not comprise the NQO1*2 (+/+) polymorphism and the lowered QH2:Q redox status exhibited by heterozygous individuals (i.e., individuals whose genome comprises one copy of the NQO1*2 polymorphism or +/−).

### DETAILED DESCRIPTION OF THE INVENTION

The location and sequence of the human gene encoding NQO1 enzyme has been described by others, and has a polymorphism that occurs in that gene. The polymorphism is a C-to-T base change at position 609 of the human cDNA encoding NQO1, which results in a proline to serine amino acid residue change at amino acid residue 187 of the NQO1 protein. This polymorphism is designated the NQO1*2 polymorphism. Others have linked the NQO1*2 polymorphism with pediatric hematologic neoplasms. (e.g., Kracht, Haematologica, 2004; 89: 1492-1497).

It was recognized that the NQO1*2 polymorphism significantly reduces the enzymatic activity of NQO1, and the NQO1*2 form of the enzyme appears to be more quickly degraded by the cellular proteosomal system than is the wild type (NQO1*1) form of the enzyme. Cells and tissues of a heterozygous individual (i.e., one who harbors both an NQO1*1 polymorphism and an NQO1*2 polymorphism) can be expected to have not more than about half the NQO1 enzymatic activity of that of an individual who is homozygous for the wild type (i.e., two copies of NQO1*1 designated NQO1*1/*1 or +/+). Likewise, individuals who are homozygous for the polymorphism (i.e., an individual with two copies of NQO1*2 polymorphism designated NQO1*2/*2 or −/−) can be expected to have significantly decreased, if any, NQO1 activity, relative to a person who is homozygous for the wild type form. Siegel reports that in genotype-phenotype studies, individuals who are homozygous for the NQO1*2 polymorphism exhibit decreased capacity to protect against oxidative damage. Accordingly, the detoxification, chemoprotective and chemopreventive attributes of NQO1 do not inure to these individuals. (Siegel, D., et al., Rapid Polyubiquitination and Proteasomal Degradation of a Mutant Form of NAD(P)H:Quinone Oxidoreductase 1, Mol. Pharmacol. 2001; 59:263-68, citing Traver, et al., Characterization of a Polymorphism in NAD(PH):quinine oxidoreductase (DT-diaphorase), Cancer Res. 1997; 75:69-75).

The studies relating to NQO1 enzymatic activity as a function of the presence of one or two NQO1*2 polymorphisms do not explore the effect of reduced enzymatic activity relative to the QH2:Q redox status of an individual. The present invention provides an indicator of the QH2:Q redox status of an individual (a measure which is difficult to obtain) by analysis of the individual's genome for presence or absence of the NQO1*2 polymorphism (an analysis which is readily performed).

The NQO1*2 polymorphism is relatively common in the human population. For example, approximately 40% of Caucasians are NQO1*1/*2 heterozygotes and about 4% are NQO1*2/*2 homozygotes. (Kelsey, K. T., et al., Ethnic Variation in the Prevalence of a Common NAD(P)H:quinine oxidoreductase Polymorphism and its Implications For Anti-cancer Chemotherapy, Br. J. Cancer 1997; 76:852-54).

In experiments performed at the inventors' direction in support of this disclosure, archived blood samples obtained from individuals were tested. The CoQ redox ratio (i.e., the ratio of reduced to oxidized CoQ) was assessed in each sample. Occurrence of the NQO1*2 in the genomes of the individuals from whom the blood samples were obtained were also assessed. A statistically significant association between the occurrence of the NQO1*2 polymorphism and a decreased CoQ (namely, QH2:Q) redox ratio was observed. The summary provided on Table 3 illustrates this conclusion.

These observations indicate that individuals who harbor at least one NQO1*2 polymorphism exhibit a physiologically significant deficit in their ability to convert oxidized CoQ to its physiological useful reduced state. It was previously unknown whether a decrease in NQO1 activity associated with occurrence of the NQO1*2 polymorphism in an individual's genome would have any effect (physiologically significant or not) on the redox state of CoQ in cells and tissues of the individual.

A conclusion which can be drawn from the results presented herein is that an individual who harbors one or two NQO1*2 polymorphisms can benefit from nutritional supplementation with CoQ, and more particularly, reduced CoQ. An NQO1*2 homozygote is likely to be more in need of nutritional supplementation than a heterozygote. Furthermore, in view of the physiological role of NQO1 (i.e., reducing oxidized CoQ), it can be concluded that nutritional supplementation for an individual who harbors one or two NQO1*2 polymorphisms will be more effective if the supplement is the reduced form of CoQ (ubiquinol or QH2). Ubiquinol has a higher bioavailability than the oxidized form of CoQ (ubiquinone or Q). To that end, orally administered compositions directed to increasing the bioavailability of CoQ are disclosed in the art. (Chopra, R., 2004, U.S. Pat. No. 6,740,338).

It is recognized that supplementation of an individual's nutrition with CoQ can increase body levels of CoQ. When oxidative stress is low, a significant fraction of the CoQ can be maintained in its reduced form in the body. Faced with oxidative stress, the antioxidant capacity of the pool of reduced CoQ is used up (generating oxidized CoQ). Under such conditions, the rate at which individuals who harbor one NQO1*2 polymorphism recycle the reduced form will be less than that of NQO1*1 homozygotes and this rate will be much lower for NQO1*2 homozygotes. With a larger pre-stress pool of CoQ, a greater amount of reduced CoQ is available. For that reason, the oxidative stress can be better tolerated by an individual who harbors one or two NQO1*2 polymorphisms if that individual's diet is supplemented with CoQ prior to the stress.

Supplementation of an individual's nutrition with CoQ in a reduced form (ubiquinol) form can increase body levels of reduced CoQ. In individuals who have significantly impaired (or no) NQO1 activity, nutritional supplementation with reduced CoQ can restore physiological levels of reduced CoQ. Thus, supplementation with reduced CoQ can be recommended for individuals with severely impaired levels of NQO1 activity and who imminently expect to experience oxidative stress (e.g., individuals preparing for strenuous exercise).

It is recognized that CoQ is not a single molecule, but rather a family of molecules having a benzoquinone moiety linked with multiple isoprene units (ordinarily 9 or 10 isoprene units in humans; thus CoQ is often referred to as CoQ9—when 9 isoprene units are present—or CoQ10—when 10 isoprene units are present). The term CoQ as used herein incorporates all forms of coenzyme Q, unless specified otherwise. Furthermore, it is recognized that CoQ and the NQO1 enzyme exhibit analogous functions in non-human mammals, and the observations made herein are equally applicable to such animals.

Any of a wide variety of commercially-available or readily-designable kits and methods can be used to detect occurrence of the NQO1*2 polymorphism in an individual. Examples of suitable methods include gene sequencing, amplification of a portion of a gene (or RNA) that includes the polymorphic residue, immunological detection NQO1*2 protein, and nucleic acid hybridization-based methods. Each of these methods is known in the art and can be employed to assess occurrence of an NQO1*2 polymorphism in an individual with little, if any, experimentation required.

### 1 DEFINITIONS

As used in this disclosure, the following terms have the meanings associated with them in this section.

A “polymorphism” is a gene in one of the alternate forms of a portion of the gene that are known to occur in the human population. For example, many genes are known to exhibit single nucleotide polymorphic forms, whereby the identity of a single nucleotide residue of the gene differs among the forms. Each of the polymorphic forms represents a single polymorphism, as the term is used herein. Other known polymorphic forms include alternative forms in which multiple consecutive or closely-spaced, non-consecutive nucleotide residues vary in sequence, forms which differ by the presence or absence of a single nucleotide residue or a small number of nucleotide residues, also known respectively as insertion polymorphisms and deletion polymorphisms, and forms which exhibit different mRNA splicing patterns.

A “single nucleotide polymorphism” (“SNP”) is one of the alternative forms of a portion of a gene that vary only in the identity of a single nucleotide residue in that portion.

“Toxic oxygen species” include, in approximate order of decreasing reactivity, hydroxyl radicals, superoxide radicals, nitric oxide, peroxy nitrate (ONOO⁻; the product of reaction between nitric oxide and a superoxide radical), and hydrogen peroxide. Ordinary diatomic oxygen is not a toxic oxygen species, as the term is used herein.

The “NQO1*2” polymorphism is a C-to-T base change at position 609 of the human cDNA encoding NQO1, which results in a proline to serine amino acid residue change at amino acid residue 187 of the NQO1 protein.

“Oxidative damage” refers to a chemical reaction of a normal cellular component (e.g., DNA, a protein, or a lipid) with a toxic oxygen species, whereby at least one normal function of the component is inhibited or eliminated. The terms “oxidative damage” and “oxidative stress” are used interchangeably herein.

An “instructional material” is a publication, recording, diagram, educational material, or any other medium of expression which can be used to communicate how to use a method or kit described herein or the assessment results of the inventors' methods. Alternatively, an instructional material may be advisory or educational. The instructional material can be used cooperatively with the kits or methods described herein.

A “control” is an actual or hypothetical individual whose genome does not comprise a single polymorphism which is assessed by the methods disclosed herein or an actual or hypothetical individual whose genome comprises a known quantity of polymorphisms assessed by the methods disclosed herein.

A “biological sample” is a biological sample including, for example and without limitation, blood, tissue, or urine, taken or collected from an individual for analysis, testing, storage, or assessment purposes.

The term “phenotype” as used herein refers to the accepted meaning of that term which includes not only readily observable physical characteristics of an organism, but also includes measurable biochemical or physiological traits of an organism such as blood type or QH2:Q redox status.

A “statin” is a HMG-CoA reductase inhibitor broadly known as a lipid-lowering drug (or hypolipidemic agent) which is typically employed to lower cholesterol by inhibiting the enzyme HMG-CoA reductase in the mevalonate pathway of cholesterol synthesis.

An “individual” is a singular human subject.

The expression “QH2:Q” represents the ratio between the reduced form of CoQ (ubiquinol or QH2) and the oxidized form of CoQ (ubiquinone or Q).

An “effective dose” is the median dose that produces the desired therapeutic effect of the compound or composition administered. A minimum effective dose or a minimum therapeutically effective dose of a compound or composition can be estimated in a number of ways. For example, treatment doses can be compared to placebo doses to determine the lowest dosage at which the effect of the compound is statistically significant. (Filloon, T. G., et al., “Estimating the Minimum Therapeutically Effective Dose of a Compound via Regression Modelling and Percentile Estimation,” Stat. Med. 1995 May; 14(9-10): 925-32). As used herein, effective dose or “therapeutically effective dose” describes an amount of compound or composition which may be used to produce a favorable result according to the present invention.

The term “compensatory pathway,” as used in this disclosure, refers to an enzymatic pathway (other than conversion by NQO1), described by the inventors or others, which results in the reduction of CoQ. This occurrence is sometimes referred to in the art as gene compensation.

The expression “+/+” represents the genotype of an individual who is homozygous for an advantaged (+) allele. In the case of the present invention, the advantaged allele is NQO1*L (or wild-type) form of the gene encoding NQO1. This genotype is also designated herein as “NQO1*1/*1.” A visual indicator of this genotype for purposes of preparing an instructional material utilizes color. Using the familiar three-color indicator green/yellow/red, this genotype can be identified by the color green. Other visual representations may be employed without departing from the spirit of the present invention.

The expression “−/−” represents the genotype of an individual who is homozygous for the disadvantaged (−) allele. In the case of the present invention, the disadvantaged allele is the NQO1*2 polymorphic form of the gene encoding NQO1. This genotype is also designated herein as “NQO1*2/*2.” A visual indicator of this genotype for purposes of preparing an instructional material utilizes color. Using the familiar three-color indicator green/yellow/red, this genotype can be identified by the color red. Other visual representations may be employed without departing from the spirit of the present invention.

The expression “+/−” represents the genotype of an individual who is heterozygous; namely, carrying one advantaged (+) and one disadvantaged (−) allele (i.e., NQO1*1/*2). A visual indicator of this genotype for purposes of preparing an instructional material utilizes color. Using the familiar three-color indicator green/yellow/red, this genotype can be identified by the color yellow. Other visual representations may be employed without departing from the spirit of the present invention.

The term “allele” refers to coding sequences found at various polymorphic sites in an individual's genome.

A “composition” is a compound comprising a nutritional (whether customized, i.e., a genetically-guided nutrigenomic product, or generic) supplement which may be ingested or applied topically.

An “intervention” is advising or employing consumption or use of a nutritional supplement or composition, advising or employing a nutritional supplement or composition, or advising or undergoing heightened medical monitoring.

A “nutrigenomic” composition is a customized nutritional supplement formulated to address an individual's supplementation needs based on the individual's predisposition to certain conditions as determined by assessment of the individual's genome. Nutrigenomic compositions are non-pharmaceutical; they are, however, compensatory and can be used to alleviate, inhibit, or prevent a disease state.

### 2. STATINS

HMG-CoA reductase inhibitors, known as statins, block production of mevalonate that functions in the synthesis of cholesterol. Mevalonate is a precursor of coenzyme Q10 (CoQ10). Thus, collateral with the reduction of cholesterol by statin treatment is a reduction in CoQ10 synthesis by the body.

A known complication of statin usage and concomitant CoQ10 lowering is progressive muscle weakening or myopathy, which can lead to the more severe form of muscle injury known as rhabdomyolysis. A decrease in CoQ10 levels affected by statin administration has other potential side effects as well. CoQ10 plays a critical role in the respiratory chain of mitochondria. CoQ10 also acts as a protective agent against oxidative stress, for example, by inhibiting oxidation of lipoproteins that can contribute to the development of atherosclerosis. Thus, CoQ10 is protective against various forms of heart failure and other cardiovascular diseases. It is also protective against neuronal damage.

Supplementation with CoQ10 compensates for its decreased synthesis by the body resulting from statin usage. There are two forms of CoQ10, the reduced form (ubiquinol) and the oxidized form (ubiquinone). The form of CoQ10 required to act as an antioxidant is ubiquinol. Individuals with the disadvantaged polymorphism disclosed herein in the NQO1 (NAD(P)H:quinone oxidoeductase 1) gene (at position 609, C→T, causing a proline to serine substitution) are less able to convert the oxidized form of COQ10 (ubiquinone) to its useful reduced form (ubiquinol). Such individuals would benefit more from supplementation with ubiquinol than with ubiquinone, and the kits and methods described herein can be used to identify individuals who are either homozygous or heterozygous for the disadvantaged polymorphism. Homozygous individuals in particular will benefit from supplementation with reduced (but often more expensive) ubiquinol form of CoQ10. For this reason, the inventors' methods are particularly relevant to individuals undergoing treatment with a statin drug.

In view of the known inhibition of COQ10 production that occurs in individuals undergoing statin treatment (i.e., patients who have recently begun statins or have taken them for an extended period of time), the kits and methods described herein are particularly suitable for use by or for such individuals.

### 3. BEST MODE OF PRACTICING THE INVENTION

The methods relating to assessing the advisability that an individual should employ CoQ, and more particularly QH2, supplementation is best practiced by:
(a) obtaining a biological sample from the individual using, for example, a buccal swab;
(b) assessing the individual's genome for absence or presence of one or two copies of the NQO1*2 polymorphism;
(c) predicting the QH2:Q redox status of the individual based on absence or presence of one or two copies of the NQO1*2 polymorphism;
(d) advising an individual whose genome comprises one or two copies of the NQO1*2 polymorphism to supplement their nutrition with CoQ and, more preferably, the reduced form of CoQ (ubiquinol);
(e) providing the individual with an instructional material supplying information relative to the genomic assessment and advised nutritional support; and
(f) applying the methods to individuals undergoing treatment with a statin.

Predicting the QH2:Q redox status of the individual by the aforementioned method further discloses whether the individual is predisposed to lowered QH2:Q redox status. Individuals whose genome comprises one or two copies of the NQO1*2 polymorphism are predisposed to lowered QH2:Q redox status compared to the optimal QH2:Q redox status exhibited by individuals whose genome does not comprise the NQO1*2 polymorphpism.

Studies purporting to indicate appropriate therapeutically effective doses of CoQ to increase the QH2:Q redox ratio have been performed by others in the field. (e.g., U.S. Pat. No. 6,184,255 to Mae, 2001). Thus, appropriate therapeutically effective doses of CoQ can be determined by one skilled in the art without undue experimentation.

### 4. DESCRIPTION OF THE PREFERRED EMBODIMENTS

### a. First Preferred Embodiment

The invention relates to methods for assessing the genome of an individual for presence or absence of the NQO1*2 polymorphism for the purpose of predicting the QH2:Q redox status of the individual. The examples given below are for illustrative purposes only and are not intended by the inventors to limit the scope of the invention.

In the first preferred embodiment of the invention, the method comprises obtaining a biological sample from the individual and assessing the individual's genome for the NQO1*2 polymorphism. The method by which the assessment is performed is not critical. For example, occurrence of the polymorphisms can be assessed using a method that includes contacting a nucleic acid derived from the individual's genome with a first oligonucleotide. The first oligonucleotide can be one that anneals with higher stringency with the disadvantaged polymorphism than with a corresponding advantaged polymorphism. Annealing of the first oligonucleotide and the nucleic acid can be assessed, and such annealing is an indication that the individual's genome comprises the disadvantaged polymorphism. Use of an oligonucleotide has the advantage that the oligonucleotide can be attached to a support using routine methods, and that a plurality of oligonucleotides can be attached to the same support, to allow simultaneous detection of multiple polymorphisms. If a second oligonucleotide which anneals with higher stringency with an advantaged polymorphism than with a corresponding disadvantaged polymorphism is used, then the allelic content (i.e., heterozygous or homozygous for one or the other polymorphic form) of the individual's genome can be determined. Detection of polymorphic sequences can be simplified by using labeled oligonucleotides, such as molecular beacon oligonucleotides. Alternatively, as mentioned above, there are a number of commercially-available or readily-designable kits and methods can be used to detect occurrence of the NQO1*2 polymorphism in an individual, including gene sequencing, amplification of a portion of a gene (or RNA) that includes the polymorphic residue, immunological detection of the NQO1*2 protein, and nucleic acid hybridization-based methods. Each of these methods is known in the art and can be employed to assess occurrence of an NQO1*2 polymorphism in an individual with little, if any, experimentation required. Furthermore, each of these methods is suitable in connection with the disclosed methods.

Once the number of NQO1*2 polymorphisms present in the individual's genome is determined (whether zero, one or two), the QH2:Q redox status of that individual may be predicted in accordance with the inventors' methods. An individual whose genome does not comprise the NQO1*2 polymorphism is advantaged and is expected to exhibit optimal QH2:Q redox status. The experiments performed in support of this disclosure suggest that an optimal QH2:Q redox status is 16.9±2.24 μmol/l. An individual whose genome comprises at least one copy of the NQO1*2 polymorphism is expected to exhibit a lower QH2:Q redox status than an advantaged individual (i.e., an individual who does not harbor the NQO1*2 polymorphism). The experiments performed in support of this disclosure suggest that the lowered QH2:Q redox status exhibited by an individual with one NQO1*2 polymorphism is 11.87±1.06 μmol/l. The data obtained by the experiments performed in support hereof discloses, significantly, that the lowest QH2:Q redox status observed in an advantaged (NQO1*1*1 or +/+) individual-12.73 μmol/l—is higher than the highest QH2:Q redox status observed in a heterozygous (NQO1*1/*2 or +/−) individual, with that measure being 12.38 μmol/l.

After the expected QH2:Q redox status of the individual is predicted based on assessment of the individual's genome for the NQO1*2 polymorphism, a phenotypic designation can be assigned to the individual. An individual whose genome does not comprise a single copy of the NQO1*2 polymorphism is phenotypically advantaged for QH2:Q redox status. An individual whose genome comprises one copy of the NQO1*2 polymorphism exhibits a phenotypically lower QH2:Q redox status than an advantaged individual. And, an individual whose genome comprises two copies of the NQO1*2 polymorphism exhibits phenotypically diminished (or significantly lower) QH2:Q redox status. Words of similar import to advantaged, optimal, lower, diminished, or significantly lower may be used interchangeably without departing from the essence of the present invention.

Thereafter, an individual whose genome comprises one or two copies of the NQO1*2 polymorphism is advised to supplement the nutrition of that individual with CoQ or otherwise employ a CoQ composition. Most preferably, though not necessarily, the amount of CoQ advised, administered, or employed is effective to increase the QH2:Q redox status of that individual to the optimal range exhibited by an individual whose genome does not comprise the NQO1*2 polymorphism. Supplementation dosage ranges in that connection are known. Further, it is most preferable to advise supplementation with the reduced form of CoQ (ubiquinol) for its bioavailability.

Supplementation with CoQ (and most preferably ubiquinol) will alleviate enzymatic pathways, compensatory pathways, which compensate for reduced (or null) NQO1 enzymatic activity.

This method is particularly relevant to individuals undergoing treatment with a statin drug for the reason that statins are known to significantly reduce plasma concentrations of essential CoQ in individuals being treated with statins.

### b. Second Preferred Embodiment

In another aspect, the invention relates to a method of determining whether an individual is predisposed to a lowered QH2:Q redox status. The method comprises obtaining a biological sample from the individual and assessing the individual's genome for the NQO1*2 polymorphism. The method by which the assessment is performed is not critical. Each of the assessment methods described immediately above in connection with the first preferred embodiment are suitable. Presence of one or two copies of the NQO1*2 polymorphism in the individual's genome is an indication that the individual is predisposed to a lowered QH2:Q redox status. Accordingly, the method further comprises advising an individual whose genome comprises at least one copy of the NQO1*2 polymorphism to supplement the nutrition of that individual with CoQ or otherwise employ a CoQ composition. Most preferably, although not necessarily, the amount of CoQ advised, administered, or employed is effective to increase the QH2:Q redox status of that individual to the optimal range exhibited by an individual whose genome does not comprise the NQO1*2 polymorphism. Supplementation dosage ranges in that connection are known. Further, it is most preferable to advise supplementation with the reduced form of CoQ (ubiquinol) for its bioavailability.

This method is particularly relevant to individuals undergoing treatment with a statin drug for the reason that statins are known to significantly reduce plasma concentrations of essential CoQ in individuals being treated with statins.

In connection with this second preferred embodiment, the QH2:Q phenotype of the individual can further be determined. An individual whose genome does not comprise a single copy of the NQO1*2 polymorphism is phenotypically advantaged for QH2:Q redox status. An individual whose genome comprises one copy of the NQO1*2 polymorphism exhibits a phenotypically lower QH2:Q redox status than an advantaged individual. And, an individual whose genome comprises two copies of the NQO1*2 polymorphism exhibits phenotypically diminished (or significantly lower) QH2:Q redox status. Words of similar import to advantaged, optimal, lower, diminished, or significantly lower may be used interchangeably without departing from the essence of the present invention.

Further to this second preferred embodiment, an instructional material may be provided to the individual advising the individual of the number of NQO1*2 polymorphisms present in the individual's genome (whether zero, one or two) and advising an individual whose genome comprises one or more NQO1*2 polymorphisms to supplement the nutrition of that individual with CoQ in accordance with this method.

### c. Third Preferred Embodiment

In yet another preferred embodiment, the invention relates to assessing whether an individual will benefit from nutritional supplementation with CoQ or, more particularly, ubiquinol. The method comprises obtaining a biological sample from the individual and assessing the individual's genome for the NQO1*2 polymorphism. The method by which the assessment is performed is not critical. Each of the assessment methods described above in connection with the first preferred embodiment are suitable. Presence of one or two copies of the NQO1*2 polymorphism in the individual's genome is an indication that the individual will benefit from the supplementation or implementation of a CoQ composition.

In connection with this third preferred embodiment, the QH2:Q phenotype of the individual can further be determined. An individual whose genome does not comprise a single copy of the NQO1*2 polymorphism is phenotypically advantaged for QH2:Q redox status. An individual whose genome comprises one copy of the NQO1*2 polymorphism exhibits a phenotypically lower QH2:Q redox status. And, an individual whose genome comprises two copies of the NQO1*2 polymorphism exhibits phenotypically diminished (or significantly lower) QH2:Q redox status. Words of similar import to advantaged, optimal, lower, diminished, or significantly lower may be used interchangeably without departing from the essence of the present invention.

Further to this third preferred embodiment, an instructional material may be provided to the individual advising the individual of the number of NQO1*2 polymorphisms present in the individual's genome (whether zero, one or two) and advising an individual whose genome comprises one or more NQO1*2 polymorphisms to supplement the nutrition of that individual with CoQ. Most preferably, although not necessarily, the amount of CoQ advised, administered, or employed is effective to increase the QH2:Q redox status of that individual to the optimal range exhibited by an individual whose genome does not comprise the NQO1*2 polymorphism. Supplementation dosage ranges in that connection are known. Further, it is most preferable to advise supplementation with the reduced form of CoQ (ubiquinol) for its bioavailability.

### d. Fourth Preferred Embodiment

The methods described in connection with the first preferred embodiment may also be used to determine the QH2:Q redox status of an individual.

### 5. EXPERIMENTS PERFORMED IN SUPPORT OF THE DISCLOSURE

Empirical data relating to the experiments performed in support of this disclosure is contained in Tables 1, 2 and 3.

a. Collection of a Biological Sample for Genotyping

The kit described in U.S. Pat. No. 6,291,171 (Ricciardi, et al.) was employed to collect a biological sample from each of the study participants. This biological sample (specifically oral buccal mucosa or cheek cells), as a first step in the experiments performed in support of this disclosure, was assessed to determine the genotype of the individual volunteer study participants.

b. Genotyping

The genotype of each of the study participants was assessed and the absence or presence of one or two copies of the NQO1*2 polymorphism was determined using a commercially-available test in control conditions which produces reliable results. The genotype of each individual was immediately recorded.

c. Collection of a Biological Sample for Assessment of QH2:Q Redox Status

Contemporaneous with the collection of the biological sample for genotype assessment, a whole blood sample was collected from each volunteer study participant and immediately frozen to stop the cycle of CoQ oxidation and reduction. The sample was transported (in frozen state) to the facility which performed the QH2:Q redox status assessment.

d. QH2:Q Redox Status Assessment

Electrochemical detection (HPLC-EC), which allows for detection of QH2 and Q in the same sample, was employed in the experiment performed in support of this disclosure to determine levels of QH2 and Q in each sample obtained. HPLC-EC is considered the most sensitive method for measuring CoQ.

e. Reporting

The results of this experiment were immediately recorded. The data collected in connection with the experiment described supports the operability of the invention disclosed herein. Accordingly, the inventors' methods can be carried out without undue experimentation.

These observations indicate that individuals who harbor at least one NQO1*2 polymorphism exhibit a physiologically significant deficit in their ability to convert oxidized CoQ to its physiological useful reduced state. It was previously unknown whether a decrease in NQO1 activity associated with occurrence of the NQO1*2 polymorphism in an individual's genome would have any effect (physiologically significant or not) on the redox state of CoQ in cells and tissues of the individual.

The disclosure of every patent, patent application, and publication cited herein is hereby incorporated by reference in its entirety.

While this subject matter has been disclosed with reference to specific embodiments, it is apparent that other embodiments and variations can be devised by others skilled in the art without departing from the true spirit and scope of the subject matter described herein. The appended claims include such embodiments and equivalent variations.

### DRAWINGS

TABLE 1
STUDY PARTICICPANT GENOTYPES

| Study | | | |
|---|---|---|---|
| Participant | | Participant NQO1 | Genotype |
| No. | Sample ID No. | Genotype | Designation |
| 1 | 010100003316 | CT (NQO1*1/*2) | (+/−) (Yellow) |
| 2 | 010100003319 | CC (NQO1*1/*1) | (+/+) (Green) |
| 3 | 010100003322 | CT (NQO1*1/*2) | (+/−) (Yellow) |
| 4 | 010100003327 | CC (NQO1*1/*1) | (+/+) (Green) |
| 5 | 010100003329 | CC (NQO1*1/*1) | (+/+) (Green) |
| 6 | 010100003330 | CC (NQO1*1/*1) | (+/+) (Green) |
| 7 | 010100003332 | CC (NQO1*1/*1) | (+/+) (Green) |
| 8 | 010100003333 | CC (NQO1*1/*1) | (+/+) (Green) |
| 9 | 010100003335 | CC (NQO1*1/*1) | (+/+) (Green) |
| 10 | 010100003339 | CC (NQO1*1/*1) | (+/+) (Green) |
| 11 | 010100003342 | CC (NQO1*1/*1) | (+/+) (Green) |
| 12 | 010100003343 | CC (NQO1*1/*1) | (+/+) (Green) |
| 13 | 010100003345 | CC (NQO1*1/*1) | (+/+) (Green) |
| 14 | 010100003356 | CT (NQO1*1/*2) | (+/−) (Yellow) |
| 15 | 010100003357 | CC (NQO1*1/*1) | (+/+) (Green) |
| 16 | 010100003358 | CT (NQO1*1/*2) | (+/−) (Yellow) |

SUMMARY

| | | |
|---|---|---|
| CC (+/+) (Green) | 12 | 75% |
| CT (+/−) (Yellow) | 4 | 25% |

TABLE 2
PARTICIPANT DEMOGRAPHICS
As of Mar. 30, 2004

| Participant ID | Sample ID | Age | Gender |
|---|---|---|---|
| 3319 | 010100003319 | 41 | F |
| 3327 | 010100003327 | 40 | F |
| 3329 | 010100003329 | 41 | F |
| 3330 | 010100003330 | 39 | F |
| 3332 | 010100003332 | 37 | F |
| 3333 | 010100003333 | 37 | F |
| 3335 | 010100003335 | 39 | F |
| 3339 | 010100003339 | 49 | F |
| 3342 | 010100003342 | 54 | F |
| 3343 | 010100003343 | 52 | F |
| 3345 | 010100003345 | 43 | F |
| 3357 | 010100003357 | 45 | F |
| 3358 | 010100003358 | 53 | F |
| 3356 | 010100003356 | 39 | F |
| 3322 | 010100003322 | 54 | F |
| 3316 | 010100003316 | 46 | F |
| | SUMMARY | Mean Age = 44.31 y | F = 100% |

TABLE 3
ASSESSMENT DATA
Assessment Performed Mar. 30, 2004

| | | | | | Redox | |
|---|---|---|---|---|---|---|
| | Cholesterol | QH2 | Q | Total CoQ | Ratio | |
| Sample ID | (mmol/l) | (μmol/l) | (μmol/l) | (μmol/l) | (QH2:Q) | Genotype |
| 010100003319 | 4.81 | 1.46 | 0.07 | 1.53 | 20.96 | (+/+) |
| 010100003327 | 6.06 | 1.38 | 0.08 | 1.46 | 16.97 | (+/+) |
| 010100003329 | 5.10 | 1.33 | 0.08 | 1.41 | 16.40 | (+/+) |
| 010100003330 | 4.00 | 0.59 | 0.05 | 0.64 | 12.73 | (+/+) |
| 010100003332 | 4.55 | 0.99 | 0.06 | 1.05 | 17.17 | (+/+) |
| 010100003333 | 4.81 | 0.73 | 0.05 | 0.77 | 15.72 | (+/+) |
| 010100003335 | 5.17 | 1.01 | 0.07 | 1.08 | 14.47 | (+/+) |
| 010100003339 | 4.89 | 1.12 | 0.06 | 1.18 | 19.37 | (+/+) |
| 010100003342 | 3.72 | 0.82 | 0.05 | 0.87 | 17.72 | (+/+) |
| 010100003343 | 6.68 | 1.03 | 0.06 | 1.09 | 17.77 | (+/+) |
| 010100003345 | 4.37 | 1.08 | 0.06 | 1.13 | 18.57 | (+/+) |
| 010100003357 | 5.56 | 0.69 | 0.05 | 0.74 | 14.97 | (+/+) |
| 010100003358 | 4.68 | 1.04 | 0.09 | 1.13 | 11.23 | (+/−) |
| 010100003356 | 3.95 | 0.72 | 0.06 | 0.77 | 12.38 | (+/−) |
| 010100003322 | 4.63 | 1.06 | 0.08 | 1.14 | 13.12 | (+/−) |
| 010100003316 | 8.32 | 1.87 | 0.17 | 2.05 | 10.78 | (+/−) |

SUMMARY

| Genotype | QH2:Q | Stand. Dev. |
|---|---|---|
| (+/+) | 16.9 μmol/l | 2.24 |
| (+/−) | 11.87 μmol/l | 1.06 |

What is claimed is:

1. A method of predicting the QH2:Q redox status of an individual by assessment of the individual's genome, the method comprising obtaining a biological sample from the individual and assessing the individual's genome for presence or absence of the NQO1*2 polymorphism, whereby an individual whose genome does not comprise the NQO1*2 polymorphism is expected to exhibit optimal QH2:Q redox status and an individual whose genome comprises one or two copies of the NQO1*2 polymorphism is expected to exhibit lower QH2:Q redox status than an individual whose genome does not comprise the NQO1*2 polymorphism.

2. The method of claim 1, whereby an individual whose genome comprises two copies of the NQO1*2 polymorphism is expected to exhibit significantly lower QH2:Q redox status than an individual whose genome does not comprise the NQO1*2 polymorphism.

3. The method of claim 1, the method further comprising assigning a phenotypic designation to the individual indicating QH2:Q redox status based on the number of NQO1*2 polymorphisms present in the individual's genome.

4. The method of claim 1, further comprising advising an individual whose genome comprises one or two NQO1*2 polymorphisms to supplement the individual's nutrition with a therapeutically effective dose of coenzyme Q (CoQ) to increase the QH2:Q redox status of the individual to the optimal range exhibited by an individual whose genome does not comprise the NQO1*2 polymorphism.

5. The method of claim 4 wherein the form of CoQ is ubiquinol.

6. The method of claim 4 wherein the individual's nutrition is supplemented.

7. The method of claim 6, whereby compensatory pathways affected by reduced NQO1 enzymatic activity resulting from presence of one or two NQO1*2 polymorphisms in the individual's genome are alleviated.

8. A method of determining whether an individual is predisposed to lowered QH2:Q redox status, the method comprising obtaining a biological sample from the individual and detecting presence of at least one NQO1*2 polymorphism in the individual's genome, whereby presence of an NQO1*2 polymorphism in the individual's genome indicates that the individual is predisposed to lower QH2:Q redox status.

9. The method of claim 8, further comprising advising an individual whose genome comprises at least one NQO1*2 polymorphism to supplement the nutrition of that individual with CoQ.

10. The method of claim 9 wherein the form of CoQ is ubiquinol.

11. The method of claim 8, wherein the individual is an individual undergoing a statin treatment.

12. A method of assessing whether an individual will benefit from nutritional supplementation with CoQ, the method comprising obtaining a biological sample from the individual and assessing the individual's genome for presence or absence of the NQO1*2 polymorphism, whereby occurrence of at least one NQO1*2 polymorphism is an indication that the individual will benefit from the supplementation.

13. The method of claim 12, the method further comprising determining the QH2:Q redox status phenotype of the individual based on presence of absence of the NQO1*2 polymorphism whereby an individual with no NQO1*2 polymorphisms exhibits optimal QH2:Q redox status; an individual with no NQO1*2 polymorphism exhibits a lower QH2:Q redox status than an individual with no NQO1*2 polymorphisms; and an individual with two NQO1*2 polymorphisms exhibits significantly lower QH2:Q redox status than an individual with no NQO1*2 polymorphisms.

14. The method of claim 12, the method further comprising providing an instructional material tot he individual advising the individual of the number of NQO1*2 polymerphisms present in the individual's genome.

15. The method of claim 12, the method further comprising providing an instructional material to an individual whose genome comprises one or two NQO1*2 polymorphisms to supplement the nutrition of the individual with CoQ.

16. The method of claim 15 wherein the form of CoQ is ubiquinol.

17. The method of claim 12, further comprising advising an individual whose genome comprises one or two NQO1*2 polymorphisms to supplement the individual's nutrition with a therapeutically effective dose of CoQ to increase the QH2:Q redox status of the individual to the optimal range exhibited by an individual whoe genome does not comprise the NQO1*2 polymorphism.

18. The method of claim 17 wherein the form of CoQ is ubiquinol.

19. The method of claim 17, whereby the individual's nutrition is supplemented.

20. The method of claim 19, whereby compensatory pathways affected by reduced NQO1 enzymatic activity resulting from presence of one or two NQO1*2 polymorphisms in the individual's genome are alleviated.

21. The method of claim 12, wherein the individual is an individual undergoing a statin treatment.

22. The method of claim 21, whereby supplementation of the individual's nutrition with CoQ is advised.

23. The method of claim 22 wherein the form of CoQ is ubiquinol.

24. The method of claim 1, wherein the individual is undergoing a statin treatment.

25. The method of claim 1 wherein the individual is a non-human mammal.

26. The method of claim 8 wherein the individual is a non-human mammal.

27. The method of claim 1, the method further comprising advising an individual whose genome comprises one or two copies of the NQO1*2 polymorphism to employ a topically-applied composition comprising CoQ.

28. The method of claim 28 wherein the form of CoQ is ubiquinol.

29. The method of claim 8, the method further comprising advising an individual whose genome comprises one or two copies of the NQO1*2 polymorphism to employ a topically-applied composition comprising CoQ.

30. The method of claim 29 wherein the form of CoQ is ubiquinol.

31. The method of claim 12, the method further comprising advising an individual whose genome comprises one or two copies of the NQO1*2 polymorphism to employ a topically-applied composition comprising CoQ.

32. The method of claim 31 wherein the form of CoQ is ubiquinol.

33. A method of determining an individual's QH2:Q redox status phenotype by assessment of the individual's genome, the method comprising obtaining a biological sample from the individual and assessing the individual's genome for presence or absence of the NQO1*2 polymorphism, whereby an individual whose genome does not comprise the NQO1*2 polymorphism is phenotypically advantaged; and individual whose genome comprises one NQO1*2 polymorphism exhibits phenotypically lower QH2:Q redox status than a phenotypically advantaged individual, and an individual whose genome comprises two copies of the NQO1*2 polymorphism exhibits phenotypically diminished QH2:Q redox status compared to a phenotypically advantaged individual.

34. The method of claim 33, further comprising advising an individual whose genome comprises at least one NQO1*2 polymorphism to supplement the nutrition of that individual with CoQ.

35. The method of claim 34 wherein the form of CoQ is ubiquinol.

36. The method of claim 35, wherein the individual is undergoing a statin treatment.

37. The method of claim 33, further comprising advising an individual whose genome comprises one or two NQO1*2 polymorphisms to supplement the individual's nutrition with a therapeutically effective dose of CoQ to increase the QH2:Q redox status of the individual to the optimal range exhibited by an individual whose genome does not comprise the NQO1*2 polymorphism.

38. The method of claim 37 wherein the form of CoQ is ubiquinol.

39. The method of claim 37 wherein the individual's nutrition is supplemented.

40. The method of claim 39, whereby compensatory pathways affected by reduced NQO1 enzymatic activity resulting from presence of one or two NQO1*2 polymorphisms in the individual's genome are alleviated.

41. The method of claim 33 wherein the individual is a non-human mammal.

42. The method of claim 3, whereby an individual whose genome does not comprise the NQO1*2 polymorphism is phenotypically advantaged; an individual whose genome comprises one NQO1*2 polymorphism exhibits phenotypically lower QH2:Q redox status than a phenotypically advantaged individual, and an individual whose genome comprises two copies of the NQO1*2 polymorphism exhibits phenotypically diminished QH2:Q redox status compared to a phenotypically advantaged individual.

43. The method of claim 1, the method further comprising providing an instructional material to the individual advising the individual of the number of NQO1*2 polymorphisms present in the individual's genome.

44. The method of claim 1, the method further comprising providing an instructional material to an individual whose genome comprises one or two NQO1*2 polymorphisms to supplement the nutrition of that individual with CoQ.

45. The method of claim 44 wherein the form of CoQ is ubiquinol.
